(19) United States
(12) Patent Application Publication
(10) Pub. No.: US 2006/0088604 A1
(43) Pub. Date: Apr. 27, 2006

(54) Therapeutic ointment for treating dermatological infections and method of using same

(76) Inventor: Lisa Sisterman, White Bear Lake, MN (US)

Correspondence Address: FISH & RICHARDSON P.C., PO BOX 1022, MINNEAPOLIS, MN, 55440-1022, US

(21) Appl. No.: 11/256,753

(22) Filed: Oct. 24, 2005

Related U.S. Application Data
(60) Provisional application No. 60/621,953, filed on Oct. 25, 2004.

(51) Int. Cl. A61K 31/704; A61K 31/573; A61K 33/32

(52) U.S. Cl. 424/641; 514/35; 514/171; 514/39

(57) ABSTRACT

A therapeutic ointment that includes (a) a skin protecting agent, (b) an anti-inflammatory agent, and (c) an antibiotic composition, and a method of using the ointment to treat mammals suffering from dermatological infections.

### CROSS REFERENCE TO RELATED APPLICATIONS

This application claims priority under 35 U.S.C. §119(e) to U.S. Application No. 60/621,953, filed Oct. 25, 2004.

### TECHNICAL FIELD

This invention relates to treating mammals suffering from a dermatological infection.

### BACKGROUND

Mammals are prone to a number of dermatological infections. Horses, for example, often suffer from a dermatological infection called “scratches” that typically afflicts the skin on the pasterns and lower legs of the horse. Scratches is a very painful, recurring condition that causes intense itching and scab formation on the animal's skin. The current treatment for scratches involves repeatedly scraping the skin of the afflicted areas with a wire brush. It also involves using iodine scrubs and/or other caustic washes that create pain and burning for the horse. Because the skin in these areas is already raw and sensitive, the animal experiences intense pain and may kick or thrash, thereby injuring the person treating the animal.

### SUMMARY

A therapeutic ointment is described that effectively treats mammals suffering from dermatological infections such as scratches. The ointment includes (a) a skin protecting agent (e.g., zinc oxide), (b) an anti-inflammatory agent (e.g., a steroid such as hydrocortisone), and (c) an antibiotic composition (e.g., a triple antibiotic composition such as the combination of bacitracin zinc, neomycin, and Polymyxin B sulfate. It may also include a fungicide such as miconazole nitrate. The ointment is also useful for treating infectious conditions such as greasy heal, rain rot, and plaque.

The ointment offers many advantages. For example, unlike scraping with a wire brush, the ointment soothes, rather than inflames, the infected skin. Thus, it is more tolerable for the animal and minimizes the risk of injury to the person treating the animal. In addition, the treated skin begins to heal quickly, with a noticeable improvement resulting after only 1-2 days.

The details of one or more embodiments of the invention are set forth in the drawings and the description below. Other features, objects, and advantages of the invention will be apparent from the description and from the claims.

### DETAILED DESCRIPTION

The therapeutic ointment includes (a) a skin protecting agent, (b) an anti-inflammatory agent, and (c) an antibiotic composition. The purpose of the skin protecting agent is to protect the mammal's skin from, e.g., water, sun, chemicals, and the like while the skin is healing from the infection. An example of a useful skin protecting agent is zinc oxide.

The purpose of the anti-inflammatory agent is to reduce inflammation and itching that often accompanies infection. Examples of useful anti-inflammatory agents include steroids such as hydrocortisone.

The purpose of the antibiotic composition is to attack and eliminate the underlying cause of the infection, including bacteria and fungi. Preferably, one or more antibiotic agents are used in combination with each other to achieve the desired therapeutic effect. Examples include a triple antibiotic ointment featuring bacitracin zinc, neomycin, and Polymyxin B sulfate.

The relative amounts of the three components (skin protecting agent, anti-inflammatory agent, and antibiotic composition) are selected to give the desired therapeutic and protective effects. For example, in some embodiments, the ratio of skin protecting agent to anti-inflammatory agent to antibiotic composition is about 4:1:1, on a volume basis.

The ointment may further include a fungicide such as miconazole nitrate.

The ointment may further include a number of inactive ingredients. Examples include ointment bases (e.g., methylparaben, paraffin, petrolatum, white petrolatum, microcrystaline wax, bees wax, carnauba wax, and the like), plasticizers (e.g., triacetine, diacetyl ethylene glycol, diethyl sebacate, diethyl phthalate, dibutyl phthalate, diisopropyl adipate, dibutyl succinate, and the like), surfactants (e.g., polyoxyl stearate 40, polyoxyethylene hydrogenated castor oil, polyoxyethylene polyoxypropylene glycol, polysorbate, sucrose esters of fatty acids, and the like), stabilizers (e.g., calcium disodium edetate), antioxidants (e.g., BHA, BHT, and the like), emollients and humectants (e.g., lanolin, glycerin, aloe, and the like), rheology modifiers (e.g., talc), cod liver oil, shark liver oil, and mineral oil.

The ointment is prepared by admixing the various components, typically at room temperature. In use, the affected area is typically washed gently with a mild soap, rinsed well, and dried, after which the ointment is smeared over the area. In general, the ointment is applied once a day until the infection has healed.

### Example 1

A therapeutic composition was prepared by blending the following ingredient (all amounts are weight percent unless otherwise noted):

Active Ingredients

| | | |
|---|---|---|
| Zinc Oxide | 40% | |
| Hydrocortisone | 10 | mg |
| Bacitracin Zinc | 400 | units |
| Neomycin | 3.5 | mg |
| Polymyxin B sulfate | 5000 | units |

Inactive Ingredients

White Petrolatum, Aloe Extract, BHA, Cod Liver Oil, Lanolin, Methylparaben, Talc, Purified Water, Mineral Oil.

The ointment was applied to the skin of horses suffering from scratches. Within 1-2 days, the infected area exhibited a noticeable improvement.

A number of embodiments of the invention have been described. Nevertheless, it will be understood that various modifications may be made without departing from the spirit and scope of the invention. Accordingly, other embodiments are within the scope of the following claims.

What is claimed is:

1. A therapeutic ointment for treating dermatological infections comprising (a) a skin protecting agent, (b) an anti-inflammatory agent, and (c) an antibiotic composition.

2. A therapeutic ointment according to claim 1 wherein the skin protecting agent comprises zinc oxide.

3. A therapeutic ointment according to claim 1 wherein the anti-inflammatory agent comprises a steroid.

4. A therapeutic ointment according to claim 3 wherein the steroid comprises hydrocortisone.

5. A therapeutic ointment according to claim 1 wherein the antibiotic composition comprises a triple antibiotic composition.

6. A therapeutic ointment according to claim 5 wherein the triple antibiotic composition comprises bacitracin zinc, neomycin, and Polymyxin B sulfate.

7. A therapeutic ointment for treating dermatological infections comprising (a) zinc oxide, (b) hydrocortisone, and (c) an antibiotic composition comprising bacitracin zinc, neomycin, and Polymyxin B sulfate.

8. A method of treating a mammal suffering from a dermatological infection comprising applying to the skin of the mammal a therapeutic ointment comprising (a) a skin protecting agent, (b) an anti-inflammatory agent, and (c) an antibiotic composition.

9. A method according to claim 8 wherein the mammal is a horse.

10. A method according to claim 8 wherein the dermatological infection from which the mammal is suffering comprises scratches.

11. A method according to claim 8 wherein the dermatological infection from which the mammal is suffering comprises greasy heal.

12. A method according to claim 8 wherein the dermatological infection from which the mammal is suffering comprises rain rot.

13. A method according to claim 8 wherein the dermatological infection from which the mammal is suffering comprises plaque.

14. A method according to claim 8 wherein the skin protecting agent comprises zinc oxide.

15. A method according to claim 8 wherein the anti-inflammatory agent comprises a steroid.

16. A method according to claim 15 wherein the steroid comprises hydrocortisone.

17. A method according to claim 8 wherein the antibiotic composition comprises a triple antibiotic composition.

18. A method according to claim 17 wherein the triple antibiotic composition comprises bacitracin zinc, neomycin, and Polymyxin B sulfate.

19. A method of treating a mammal suffering from a dermatological infection comprising applying to the skin of the mammal a therapeutic ointment comprising (a) zinc oxide, (b) hydrocortisone, and (c) an antibiotic composition comprising bacitracin zinc, neomycin, and Polymyxin B sulfate.

20. A method according to claim 19 wherein the mammal is a horse and the dermatological infection from which the mammal is suffering comprises scratches.
